(19) United States
(12) Patent Application Publication
(10) Pub. No.: US 2010/0029007 A1
(43) Pub. Date: Feb. 4, 2010

(54) Biomarker in Inflammatory Diseases

(75) Inventors: Thomas Baumruken, Wien (AT); Andreas Billich, Wien (AT); Diana Mechtcheriakova, Wien (AT); Alexander Wlachos, Wien (AT)

Correspondence Address: NOVARTIS INSTITUTES FOR BIOMEDICAL RESEARCH, INC., 220 MASSACHUSETTS AVENUE, CAMBRIDGE, MA, 02139, US

(73) Assignee: NOVAARTIS AG

(21) Appl. No.: 12/445,432

(22) PCT Filed: Oct. 10, 2007
(86) PCT No.: PCT/EP07/60761

§ 371 Date: Apr. 13, 2009

(30) Foreign Application Priority Data
Oct. 11, 2006 (EP) 06122145.3

(51) Int. Cl. G01N 33/50; C07K 14/435

(52) U.S. Cl. 436/87; 530/395

(57) ABSTRACT

The use of CD1B, or CD1D, respectively as a target for a disorder selected from Systemic Lupus Erythematosus (SLE), Lupus Anticoagulant (LA) Rheumatoid Arthritis (RA) and Idiopathic Thrombocytopenic Purpura; and the use of CD1D as a target in a disorder which is Multiple Sclerosis (MS), e.g. as a biomarker; a method for preparing a diagnostic kit and a method for identifying agents that modulates a disorder which is mediated by elevated levels of CD1B, or CD1D, respectively, in case of CD1B, or CD1D, respectively selected from Systemic Lupus Erythematosus (SLE), Lupus Anticqagulant (LA) Rheumatoid Arthritis (RA) and Idiopathic Thrombocytopenic Purpura; and in case of CD1D a disorder which is Multiple Sclerosis (MS).

The present invention relates to biomarkers in inflammatory diseases, namely CD1B and CD1D as biomarkers in certain disorders.

CD1 (cluster of differentiation 1) is a family of glycoproteins expressed on the surface of various human antigen-presenting cells. They are related to the class I MHC molecules, and are involved in the presentation of lipid antigens to T cells.

CD1A, CD1B and CD1C (group 1 CD1 molecules) are expressed on cells specialized for antigen presentation; CD1D (group 2 CD1) is expressed in a wider variety of cells. Group 1 CD1 molecules have been shown to present foreign lipid antigens and specifically a number of mycobacterial cell wall components, to CD1-specific T cells. The natural antigens of group 2 CD1 are not well characterized. Group 2 CD1 molecules activate a group of T cells, known as Natural killer T cells because of their expression of NK surface markers such as CD161.

We have now surprisingly found that both, CD1B (NM_{—}001 764) and CD1D (NM_{—}001 766) levels are increased in blood samples from patients which are suffering from Systemic Lupus Erythematosus (SLE), Lupus Anticoagulant (LA) Rheumatoid Arthritis (RA) and Idiopathic Thrombocytopenic Purpura (ITP) compared with levels in healthy control individuals. Additionally it was found that CD1D (NM_{—}001 766) is increased in blood samples from patients which are suffering from Multiple Sclerosis (MS) compared with levels in healthy control individuals.

In several aspects the present invention provides

1. CD1B and/or CD1D for use, e.g., or the use of CD1B and/or CD1D, e.g. CD1B and/or CD1D in human CD3⁺ cells,

1.1 as a target in a disorder selected from Systemic Lupus Erythematosus (SLE), Lupus Anticoagulant (LA) Rheumatoid Arthritis (RA) and Idiopathic Thrombocytopenic Purpura in humans, preferably in SLE,

1.2 for diagnosing a disorder selected from Systemic Lupus Erythematosus (SLE), Lupus Anticoagulant (LA) Rheumatoid Arthritis (RA) and Idiopathic Thrombocytopenic Purpura (ITP), preferably SLE.

CD1B as used herein is known under the accession number NM_{—}001764.

CD1D as used herein is known under the accession number NM_{—}001766.

In one aspect of the present invention CD1B may be used as indicated in 1.1 or 1.2 above.

In case of CD1B a disorder is preferably SLE.

In another aspect of the present invention CD1D may be used as indicated in 1.1 or 1.2 above.

In case of CD1B a disorder is preferably MS, SLE, RA, LA or ITP, more preferably MS or SLE.

In another aspect the present invention provides CD1D for use, e.g., or the use of CD1D, e.g. CD1D in human CD3⁺ cells,

1.3 as a target in Multiple Sclerosis

1.4 for diagnosing Multiple Sclerosis.

In several other aspects the present invention further provides

2. CD1B and/or CD1D, e.g. or the use of CD1B and/or CD1D, as a biomarker, for a use as indicated under 1.1 to 1.4 above,

e.g. in a sample of an individual,

e.g. in a sample of a body fluid or a tissue sample of an individual,

CD1B and/or CD1D as indicated under any of 1. or 2 above includes CD1B and/or CD1D in CD3⁺ cells.

According to the present invention the mRNA expression levels of both, CD1B and CD1D, in CD3⁺ cells isolated from SLE patients have been found to be surprisingly elevated (4 to 5-fold, p<0.05); e.g. and CD1D levels in MS patients about 5-fold (p<0.001) in comparison with healthy control individuals. Elevated levels of CD1B and CD1D may also been found in RA, LA and ITP patients.

CD1B or CD1D as indicated herein includes CD1B or CD1D in any form, e.g. in the form of

a nucleic acid encoding CD1B, or CD1D, respectively, e.g. including a nucleic acid encoding a derivative of CD1B, or CD1D, respectively,

CD1B or CD1D protein, e.g. including protein which is a CD1B derivative, or CD1D derivative, respectively, or

CD1B or CD1B derivative secreting cells, or CD1D or CD1D derivative secreting cells, respectively,

preferably a nucleic acid encoding CD1B or CD1D.

“A derivative” of CD1B or CD1D nucleic acid or protein, e.g. in secreting cells, according to the present invention includes a fragment, a mutant, a variant, an homolog or a modification of a CD1B or CD1D protein, respectively; or of a nucleic acid encoding CD1B or CD1D, respectively, which retains, e.g. essentially, the biological function of CD1B or CD1D, respectively, e.g. which retains, e.g. essentially, the biological function of CD1B or CD1D, respectively, e.g. in dendritic cells.

CD1B or CD1D containing cells, e.g. including CD1B or CD1D producing cells, respectively, e.g. include CD3⁺ cells.

Thus, CD1B or CD1D, respectively, for use as provided by the present invention includes splice variants encoded by mRNA generated by alternative splicing of a primary transcript, amino acid mutants, posttranslational modifications, such as glycosylation and phosphorylation variants, and modifications which are covalent derivatives of CD1B, or CD1D respectively, and which retain the biological function of CD1B, or CD1D respectively, e.g. in CD3⁺ cells. Exemplary CD1B or CD1D derivatives include modifications wherein the CD1B, or CD1D respectively, protein is covalently modified by substitution, e.g. substitution originating from appropriate means, e.g. chemical or enzymatic means, by a moiety in the CD1B, or CD1D respectively, protein. Such a moiety e.g. includes one or more amino acids, e.g. naturally occurring amino acids and other than naturally occurring amino acids, and/or a detectable moiety. A detectable moiety includes an enzyme, a radioisotope, tags, toxins and genes such as oncogenes and tumour suppressor genes. CD1B, or CD1D derivatives further include naturally occurring variants of CD1B, or CD1D respectively, e.g. provided within a particular species. Such a variant may be encoded by a related gene of the same gene family, by an allelic variant of a particular gene, or represent an alternative splicing variant of the CD1B or CD1D gene.

A CD1B, or CD1D derivative as used herein also includes fragments of a nucleic acid encoding CD1B, or CD1D respectively, or of the CD1B, or CD1D protein, and comprises individual CD1B, or CD1D respectively, domains and smaller polypeptides derived from CD1B, or CD1D respectively, domains. Preferably, smaller polypeptides derived from CD1B, or CD1D, respectively, according to the present invention define a single functional activity which is characteristic of CD1B, or CD1D, respectively. Fragments may in theory be of almost any size, as long as they retain the biological characteristic of CD1B, or CD1D, respectively. Preferably, fragments will be between 12 and 210 nucleic acids in length or between 4 and 70 amino acids, respectively. Longer fragments are regarded as truncations of the full-length CD1B, or CD1D.

Derivatives of CD1B, or CD1D respectively, as used herein also comprise mutants thereof, which may contain amino acid deletions, additions or substitutions, subject to the requirement to retain the biological function of CD1B, or CD1D respectively, e.g. in CD3⁺ cells. Conservative amino acid substitutions may be made substantially without altering the nature of CD1B, or CD1D, respectively, e.g. by truncations from the 5′ or 3′ ends. Deletions and substitutions also include deletions and substitutions in fragments of CD1B, or CD1D respectively. CD1B, or CD1D, respectively, mutants may be produced from a DNA encoding CD1B, or CD1D respectively, which has been subjected to in vitro mutagenesis resulting e.g. in an addition, exchange and/or deletion of one or more amino acids in CD1B, or CD1D respectively. For example, substitutional, deletional or insertional variants of CD1B or CD1D, respectively, can be prepared by recombinant methods and screened for functional similarity to the native forms of CD1B, or CD1D respectively.

Derivatives of CD1B or CD1D as used herein also include CD1B, or CD1D respectively, homologs, preferably CD1B or CD1D homologs retain substantial homology with CD1B, or CD1D respectively. As used herein, “homology” means that CD1B, or CD1D, respectively and a CD1B, or CD1D, respectively, homolog share sufficient characteristics to retain the biological function of CD1B, or CD1D respectively, e.g. in CD3⁺ cells. Preferably, homology is used to refer to sequence identity. Thus, the derivatives of CD1B, or CD1D respectively, preferably retain substantial sequence identity with the nucleic acid sequence as indicated herein.

“Substantial homology”, where homology indicates sequence identity, means more than 50% sequence identity, preferably more than 75% sequence identity and even more preferably a sequence identity of 80% and more, e.g. 90% and more, such as 90%, 91%, 92%, 93%, 94%, 95%, 96%, 97%, 98% or 99%.

Preferably CD1B or CD1D is originating from a mammal, e.g. a human. The nucleic acid encoding CD1B, or CD1D respectively, protein preferably has the nucleic acid sequence as disclosed in literature for CD1B, or CD1D respectively, such as NM_{—}001764 for CD1B and NM_{—}001766 for CD1D.

Biomarker as used herein means that determination of CD1B, or CD1D, respectively, in elevated levels compared to healthy control individuals in a sample of an individual has been found to be an indicator for disorders selected from SLE, LA, RA and ITP, preferably SLE; e.g. and in case of CD1D additionally for MS, as such and/or is useful for monitoring the status of disorders selected from SLE, LA, RA and ITP, preferably SLE; e.g. and in case of CD1D additionally of MS.

In another aspect the present invention provides a method for diagnosing a disorders selected from SLE, LA, RA and/or ITP, preferably SLE; e.g. and MS, comprising

a) providing a sample of an individual,

b) determining the level of CD1B, or CD1D respectively, in said sample, e.g. the level of mRNA of CD1B, or CD1D respectively, in CD3⁺ cells,

c) comparing the level of CD1B, or CD1D respectively, as determined in step b) with a reference level from a sample of a healthy control individual, and

d) diagnosing in case of CD1B, or CD1D respectively, SLE, LA, RA and/or ITP, and in case of CD1D additionally MS, if the level of of CD1B, or CD1D respectively, is elevated compared with said reference level.

Elevated level as used herein includes a level which is 2-fold up to 20-fold or more increased compared with the level of a healthy control (donor) individual, such as 3-fold to 20-fold, e.g. 3-fold to 15-fold, e.g. 3-fold to 10-fold.

In another aspect the present invention provides a method for monitoring the therapeutic efficacy in the treatment of an individual with a substance which is expected to have an effect on reducing or curing SLE, LA, RA and/or ITP, preferably SLE; e.g. and in case of CD1D additionally MS, which method comprises determining the level of CD1B, or CD1D respectively, in cells, e.g. CD3⁺ cells, in a sample of said individual and comparing that level with the level of CD1B, or CD1D respectively, prior to administration of said substance.

A sample of an individual according to a use or a method of the present invention includes a sample of a body fluid or a tissue sample. A body fluid may be derived e.g. from blood, e.g. including isolated mononuclear cells, or from a blood fraction, e.g. including plasma or serum, preferably serum. A tissue sample may be a biopsy, e.g. such as a skin biopsy.

In another aspect the present invention provides the a use or a method of the present invention wherein a sample is a body fluid or a tissue sample of an individual, e.g. a body fluid may be derived from blood, e.g. isolated cells, such as CD3⁺ cells, or from a blood fraction, e.g. plasma or serum, e.g. serum; e.g. the tissue sample may be a biopsy, e.g. such as a skin biopsy.

Cells, e.g. CD3⁺ cells, from a sample of an individual may be isolated as appropriate, e.g. according, e.g. analogously, to a a method as conventional.

Detection means in cells for determining the level of CD1B, or CD1D respectively, include means as conventional, e.g. immunoassays, such as an immunodiagnostic method, an enzyme linked immunoassay (ELISAs); a fluorescence based assay, such as dissociation enhanced lanthanide fluoroimmunoassay (DELFIA), an radiometric assay or by carrying out a CD1B, or CD1D, respectively, specific Polymerase Chain Reaction (PCR); specifically detection means include a molecule which specifically recognizes CD1B, or CD1D respectively, e.g. a molecule which is directly or indirectly detectable, preferably comprising an antibody, including antibody derivatives or fragments thereof, e.g. an antibody which recognizes CD1B, or CD1D respectively, e.g. a label bearing CD1B, or CD1D, respectively, recognizing antibody.

Such label may be a conventional label, e.g. biotin or an enzyme such as alkaline phosphatase (AP), horse radish peroxidase (HRP) or peroxidase (POD) or a fluorescent molecule, e.g. a fluorescent dye, such as e.g. fluorescein isothiocyanate. Preferably the label is biotin. The label bearing molecule, e.g. the label bearing antibody, may be detected according to methods as conventional, e.g. via fluorescence measurement or enzyme detection methods.

An antibody fragment or antibody derivative includes a fragment or a derivative, e.g. chemically or enzymatically modified, of an antibody which still is capable of recognising CD1B, or CD1D, respectively.

CD1B, or CD1D, respectively, secreting cells in a sample of a body fluid of an individual, e.g. blood, may be determined by a method as conventional, e.g. by the following method:

Cells, e.g. dendritic cells may be purified, e.g. separated by a density gradient, from the sample, e.g. blood, and the purified cells obtained are stained. Anti-CD1B, or CD1D respectively, antibodies, e.g. fluorescence labeled anti-CD1B, or CD1D, antibodies, respectively, are added to the stained cell preparation, optionally after stimulation of the cells, e.g. with interleukin-4, and the level of CD1B, or CD1D, respectively, secreting cells is determined.

Optionally, CD1B, or CD1D respectively, comprised in the sample or the CD1B, or CD1D, respectively, recognizing, e.g. detectable, molecule comprised in the detection means is immobilized on a solid phase. An appropriate solid phase includes e.g. conventional solid phases used for immobilization, e.g. a plastic plate like a polystyrene or polyvinyl plate, especially a microtiter plate. Also microbeads can be used as a solid phase, e.g. coated microbeads. The solid phase can be coated with a coating material the nature of which depends e.g. on the label comprised in the detection means. The coating material should be able to bind to the label, e.g. if the label is biotin a coating material includes streptavidin, e.g. covalently bound to the solid phase.

Preferably determination of CD1B, or CD1D, respectively, in cells, e.g. CD3⁺ cells, is carried out by PCR

In another aspect the present invention provides a method for diagnosing a disorder or disease which is mediated, e.g. associated with, e.g. driven, by eleated levels of CD1B, or CD1D, resöpectively, or CD1B, or CD1D, resöpectively, activity according to the present invention wherein the level of CD1B, or CD1D respectively, in cells, e.g. in CD3⁺ cells, is determined by use of PCR.

In another aspect the present invention provides a method for the preparation of a kit comprising providing

a) a molecule which recognizes CD1B, or CD1D respectively, optionally in a labeled form,

b) instructions how to use said kit, e.g. in CD3⁺ cell isolates,

c) optionally detection means,

d) optionally a solid phase.

in case of CD1B, or CD1D respectively, for use in the diagnosis of SLE, LA, RA and/or ITP, preferably SLE; e.g. and in case of CD1D additionally MS, e.g. in a sample of an individual.

Such kit may further comprise a substantial component, e.g. including an appropriate environment of a sample to be tested and, e.g. appropriate means to determine CD1B, or CD1D, respectively, in a sample to be tested.

In a further aspect the present invention provides an assay for identifying an agent that modulates, e.g. mediates, e.g. is associated with a disorder selected from SLE, LA, RA and/or ITP, preferably SLE; e.g. and in case of CD1D additionally MS, comprising

a) determining the level of CD1B, or CD1D respectively, in a sample, e.g. in CD3⁺ cells of a sample, of an individual, in the absence and in the presence of a candidate compound which may be expected to modulate the level of CD1B, or CD1D, respectively,

b) identifying a candidate compound which modulates the level of CD1B, or CD1D respectively, as determined in step a) as an agent, e.g. and optionally

c) using such agent as a pharmaceutical in case of CD1B, or CD1D respectively, in the treatment of a disorder selected from SLE, LA, RA and/or ITP, preferably SLE; e.g. and in case of CD1D additionally MS.

In another aspect the present invention provides an assay for identifying an agent that is modulated, e.g. mediated, e.g. is associated with, elevated levels of CD1B, or CD1D, respectively, e.g. or CD1B, or CD1D, respectively, activity, comprising

a) determining the level of CD1B, or CD1D, respectively, in a sample of an individual, in the absence and in the presence of a candidate compound which is expected to modulate the level of CD1B, or CD1D, respectively,

b) identifying a candidate compound which modulates the level of CD1B, or CD1D, respectively, as determined in step a) as an agent, e.g. and, optionally

c) using such agent as a pharmaceutical in the treatment of disorders mediated by elevated levels of CD1B, or CD1D, respectively, or CD1B, or CD1D, respectively, activity,

e.g. wherein such disorder is selected from SLE, LA, RA and/or ITP, preferably SLE; e.g. and in case of CD1D additionally MS.

Preferably a candidate compound identified decreases the level of CD1B, or CD1D respectively.

The level of CD1B, or CD1D respectively, may be determined as appropriate, e.g. as described herein.

A candidate compound as described herein is a compound which may be expected to modulate the level of CD1B, or CD1D respectively, e.g. or CD1B or CD1D activity, or CD1B or CD1D secreting cells, and includes compound(s) (libraries) from which its influence on CD1B, or CD1D respectively, can be determined. Compound (libraries) include for example oligopeptides, polypeptides, proteins, antibodies, mimetics, small molecules, e.g. low molecular weight compounds (LMW's).

An agent is a candidate compound which modulates the level of the level of CD1B, or CD1D respectively, or CD1B or CD1D activity, or CD1B, or CD1D secreting cells, e.g. in cells, such as CD3⁺ cells in a sample form a patient, e.g. a blood sample, such as serum, e.g. or a skin biopsy. An agent includes oligopeptides, polypeptides, proteins, antibodies, mimetics, small molecules, e.g. low molecular weight compounds (LMW's).

In another aspect the present invention provides an agent identified by an assay or a method of the present invention.

An agent of the present invention may exhibit pharmacological activity and is therefore useful as a pharmaceutical. An agent of the present invention may show therapeutic activity, e.g. in disorders selected from SLE, LA, RA and/or ITP, preferably SLE; e.g. and in case of CD1D additionally MS, e.g. which disorders are mediated, e.g. associated with, e.g. driven by elevated CD1B, or CD1D, respectively, levels, e.g. or CD1B, or CD1D, respectively, activity.

In another aspect the present invention provides the use of an agent of the present invention as a pharmaceutical in case of CD1B, or CD1D, respectively, for the treatment of disorders selected from SLE, LA, RA and/or ITP, preferably SLE; e.g. and in case of CD1D additionally MS, e.g. which disorders are mediated, e.g. associated with, e.g. driven by elevated levels of CD1B or CD1D, respectively, or CD1B or CD1D activity.

It may reasonably be expected that elevated levels of CD1B or CD1D, respectively, are connected with of CD1B or CD1D, respectively, activity.

For pharmaceutical use an agent of the present invention for treatment includes one or more, preferably one, agent of the present invention, e.g. a combination of two or more agents of the present invention.

In another aspect the present invention provides the use of an agent of the present invention for the manufacture of a medicament for the treatment of disorders selected from SLE, LA, RA and/or ITP, preferably SLE; e.g. and in case of CD1D additionally MS, e.g. which disorders are mediated, e.g. associated with, e.g. driven by elevated levels of CD1B or CD1D, respectively, or CD1B or CD1D activity.

In another aspect the present invention provides a pharmaceutical composition comprising an agent of the present invention beside at least one pharmaceutical excipient, e.g. appropriate carrier and/or diluent, e.g. including fillers, binders, disintegrators, flow conditioners, lubricants, sugars and sweeteners, fragrances, preservatives, stabilizers, wetting agents and/or emulsifiers, solubilizers, salts for regulating osmotic pressure and/or buffers.

In another aspect the present invention provides a method for the treatment disorders of in case of CD1B or CD1D, respectively, selected from SLE, LA, RA and/or ITP, preferably SLE; e.g. and in case of CD1D additionally MS, e.g. which disorders are mediated, e.g. associated with, e.g. driven by elevated levels of CD1B or CD1D, respectively, or CD1B or CD1D activity, comprising administering an effective amount of an agent of the present invention to a subject in need of such treatment.

For such treatment, the appropriate dosage will, of course, vary depending upon, for example, the chemical nature and the pharmakokinetic data of a compound of the present invention used, the individual host, the mode of administration and the nature and severity of the conditions being treated. However, in general, for satisfactory results in larger mammals, for example humans, an indicated daily dosage includes a range

from about 0.001 g to about 1.5 g, such as 0.001 g to 1.5 g;

from about 0.01 mg/kg body weight to about 20 mg/kg body weight, such as 0.01 mg/kg body weight to 20 mg/kg body weight,

for example administered in divided doses up to four times a day.

An agent of the present invention may be administered by any conventional route, for example enterally, e.g. including nasal, buccal, rectal, oral, administration; parenterally, e.g. including intravenous, intramuscular, subcutanous administration; or topically; e.g. including epicutaneous, intranasal, intratracheal administration; via medical devices for local delivery,

e.g. stents,

e.g. in form of coated or uncoated tablets, capsules, (injectable) solutions, solid solutions, suspensions, dispersions, solid dispersions; e.g. in the form of ampoules, vials, in the form of creams, gels, pastes, inhaler powder, foams, tinctures, lip sticks, drops, sprays, or in the form of suppositories.

For topical use, e.g. including administration to the eye, satisfactory results may be obtained with local administration of a 0.5-10%, such as 1-3% concentration of active substance several times daily, e.g. 2 to 5 times daily.

An agent of the present invention may be administered in the form of a pharmaceutically acceptable salt, e.g. an acid addition salt or metal salt; or in free form; optionally in the form of a solvate. An agent of the present invention in the form of a salt may exhibit the same order of activity as an agent of the present invention in free form; optionally in the form of a solvate.

An agent of the present invention may be used for pharmaceutical treatment according to the present invention alone, or in combination with one or more other pharmaceutically active agents.

Combinations include fixed combinations, in which two or more pharmaceutically active agents are in the same formulation; kits, in which two or more pharmaceutically active agents in separate formulations are sold in the same package, e.g. with instruction for co-administration; and free combinations in which the pharmaceutically active agents are packaged separately, but instruction for simultaneous or sequential administration are given.

### DESCRIPTION OF THE FIGURES

FIG. 1

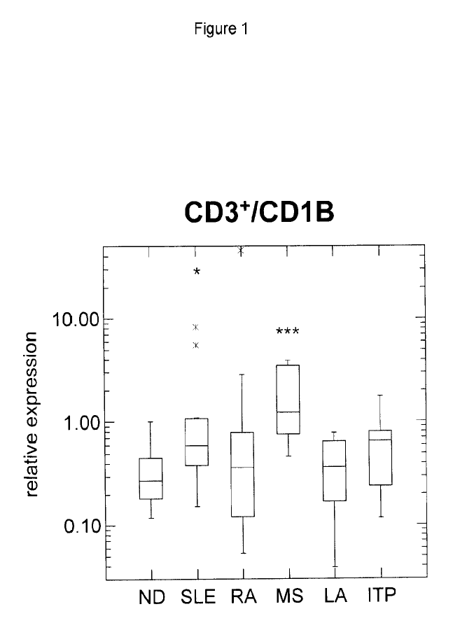

Shows the relative expression of CD1B mRNA in CD3⁺ cells of patients with autoimmune diseases compared with healthy control individuals (ND).

From FIG. 1 it is evident that in patients with autoimmune disease the expression of CD1B mRNA in CD3⁺ cells is increased compared with healthy control individuals (ND).

FIG. 2

Shows the relative expression of CD1D mRNA in CD3⁺ cells of patients with autoimmune diseases compared with healthy control individuals (ND).

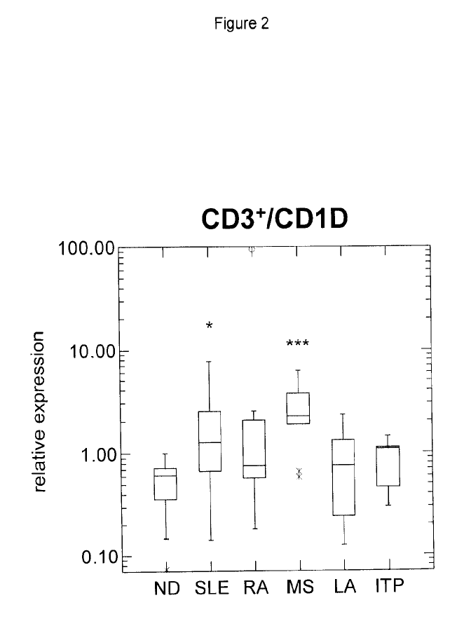

From FIG. 2 it is evident that in patients with autoimmune disease the expression of CD1D mRNA in CD3⁺ cells is increased compared with healthy control individuals (ND).

In sum, according to the present invention mRNA expression of CD1B and/or CD1D in CD3⁺ cells has been found to be associated with autoimmune disorders as indicated herein.

1. The use of CD1B and/or CD1D as a target in a disorder selected from Systemic Lupus Erythematosus (SLE), Lupus Anticoagulant (LA) Rheumatoid Arthritis (RA) and Idiopathic Thrombocytopenic Purpura; and the use of CD1D as a target in a disorder which is Multiple Sclerosis (MS).

2. The use of CD1B and/or CD1D for diagnosing a disorder selected from Systemic Lupus Erythematosus (SLE), Lupus Anticoagulant (LA) Rheumatoid Arthritis (RA) and Idiopathic Thrombocytopenic Purpura (ITP), and the use of CD1D for diagnosing Multiple Sclerosis (MS).

3. The use of CD1B or CD1D as a biomarker for a use as claimed in claim 1.

4. A method for diagnosing a disorders selected from SLE, LA, RA, ITP and MS, comprising
a) providing a sample of an individual,
b) determining the level of CD1B, or CD1D respectively, in said sample,
c) comparing the level of CD1B, or CD1D respectively, as determined in step b) with a reference level from a sample of a healthy control individual, and
d) diagnosing in case of CD1B, or CD1D respectively, SLE, LA, RA and/or ITP, and in case of CD1D additionally MS, if the level of of CD1B, or CD1D respectively, is elevated compared with said reference level.

5. A method for monitoring the therapeutic efficacy in the treatment of an individual with a substance which is expected to have an effect on reducing or curing SLE, LA, RA and/or ITP, preferably SLE; e.g. and in case of CD1D additionally MS, which method comprises determining the level of CD1B, or CD1D respectively, in cells, e.g. CD3⁺ cells, in a sample of said individual and comparing that level with the level of CD1B, or CD1D respectively, prior to administration of said substance.

6. A method for the preparation of a kit comprising providing
a) a molecule which recognizes CD1B, or CD1D respectively, optionally in a labeled form,
b) instructions how to use said kit, e.g. in CD3⁺ cell isolates,
c) optionally detection means,
d) optionally a solid phase.
in case of CD1B, or CD1D respectively, for use in the diagnosis of SLE, LA, RA and/or ITP, and in case of CD1D additionally for use in the diagnosis of MS.

7. An assay for identifying an agent that is modulated by elevated levels of CD1B, or CD1D, respectively, or CD1B, or CD1D, respectively, activity, comprising
a) determining the level of CD1B, or CD1D, respectively, in a sample of an individual, in the absence and in the presence of a candidate compound which is expected to modulate the level of CD1B, or CD1D, respectively,
b) identifying a candidate compound which modulates the level of CD1B, or CD1D, respectively, as determined in step a) as an agent, and, optionally
c) using such agent as a pharmaceutical in the treatment of disorders mediated by elevated levels of CD1B, or CD1D, respectively, or CD1B, or CD1D, respectively, activity.

8. A use, a method or an assay according to claim 1 wherein CD1B is used.

9. A use, a method or an assay according to claim 1 wherein CD1D is used.

10. The use of CD1B or CD1D as a biomarker for a use as claimed in claim 2.

11. A use, a method or an assay according to claim 2 wherein CD1B is used.

12. A use, a method or an assay according to claim 4 wherein CD1B is used.

13. A use, a method or an assay according to claim 5 wherein CD1B is used.

14. A use, a method or an assay according to claim 6 wherein CD1B is used.

15. A use, a method or an assay according to claim 7 wherein CD1B is used.

16. A use, a method or an assay according to claim 2 wherein CD1D is used.

17. A use, a method or an assay according to claim 4 wherein CD1D is used.

18. A use, a method or an assay according to claim 5 wherein CD1D is used.

19. A use, a method or an assay according to claim 6 wherein CD1D is used.

20. A use, a method or an assay according to claim 7 wherein CD1D is used.
